(19) United States
(12) Patent Application Publication
(10) Pub. No.: US 2013/0091965 A1
(43) Pub. Date: Apr. 18, 2013

(54) METHOD FOR PRODUCING A DEPOSIT OF A MATERIAL WHICH IS LOCALIZED AND HAS A DEFINED SHAPE, ON THE SURFACE OF A SUBSTRATE

(71) Applicants: Severine Vignoud, Bernin (FR); Guillaume Nonglaton, Villard-De-Lans (FR)

(72) Inventors: Severine Vignoud, Bernin (FR); Guillaume Nonglaton, Villard-De-Lans (FR)

(73) Assignee: COMMISSARIAT A L'ENERGIE ATOMIQUE ET AUX ENERGIES ALTERNATIVES, Paris (FR)

(21) Appl. No.: 13/702,298

(22) PCT Filed: Jun. 7, 2011
(86) PCT No.: PCT/EP2011/059394

§ 371 Date: Dec. 5, 2012

(30) Foreign Application Priority Data
Jun. 7, 2010 (FR) 1054450

(51) Int. Cl. B05D 1/32; G01N 33/50; B32B 33/00

(52) U.S. Cl. CPC B05D 1/32; B32B 33/00; G01N 33/50
USPC 73/866; 427/282; 427/240; 428/195.1

(57) ABSTRACT

The present invention relates to a method for producing a deposit of a material which is localized and has a defined shape on the surface of the substrate comprising the steps: (1) delimiting, by photolithography, on the surface of said substrate, at least one localized site and with a defined shape, wettable with a solution containing said material or from which said material is obtained, the areas delimiting and notably surrounding said site being non-wettable with said solution; (2) depositing on said site and said areas, said solution; whereby said material is deposited at said site.

### TECHNICAL FIELD

The present invention relates to the field of treatment of surfaces and more particularly of localized deposition of thin layers on a substrate.

Indeed, the present invention proposes a method for producing a localized deposit and with a defined shape of a material on the surface of a substrate.

The present invention also relates to the substrate on which the thin layers have been deposited in a localized way as well as to its applications in various fields such as chemical functionalization, chemical analysis and protection of substrates.

### STATE OF THE PRIOR ART

Several methods have been developed for depositing thin layers, notably of materials of the <<sol-gel>> type on a given substrate. The selection of the deposition method depends on characteristics of the substrate such as its geometry or its size and on the sought properties. The methods shown hereafter are the most used.

The method for forming coatings by immersion known as <<dip coating>> simply consists in submerging the substrate in a solution containing the <<sol>> and in removing it under highly controlled and stable conditions in order to obtain a film with regular thickness. Thus, during the upward motion of the substrate, the solution flows onto the substrate. At the end of this flow, the substrate is covered with a uniform and porous film of the <<sol-gel>> type.

The method for forming a coating by centrifugation known as <<spin coating>> is also a technique for depositing thin films. The substrate is covered with an excess of solution, before being rotated at high speed. The solution spreads out, the solvent evaporates and a homogeneous film is obtained.

The deposition method by vaporization or manual spraying known as <<spray coating>> allows projection of the sol-gel through a mask with a controlled shape. This is a simple, fast technique but the reproducibility of which is difficult to obtain without any automated airbrush.

The principle of printing by contact known as <<contact printing>> is to transfer molecules by contact between the topological patterns of a stamp and the substrate. This printing comprises the steps of (1) inking a stamp with the solution to be deposited, (2) putting the inked stamp in contact with the substrate and (3) depositing the solution of the stamp on the substrate. To do this, the inking of the stamp notably in polydimethylsiloxane (PDMS), consists, in the majority of the cases, in depositing a drop of solution on the structured surface of the stamp, and then, after an elapsed time depending on the solution, in removing it and drying it under a flow of nitrogen. The inking is a key step for <<contact printing>>; the quality and the nature of the final deposit will depend on the uniformity and on the nature of the deposit of the solution on the stamp. The inked stamp is then brought into contact with a substrate. Ideally, during this contact, only the top of the topographical patterns of the stamp comes into contact with the substrate. The molecules adsorbed at its surface are therefore transferred onto the substrate by contact if they have larger affinity with the substrate than with the stamp.

The stamp may finally be removed, thereby leaving the patterns of the ink deposited on the substrate.

The <<microdrop>> technology uses the principle of ink jet printing technology. The ejection head applied in this technology consists of a glass capillary surrounded by a piezo-electric actuator allowing ejection of the drop. By applying a voltage, the piezo-electric triggering device contracts and a pressure wave propagates in the liquid. At the outlet of the capillary, the pressure difference accelerates the liquid. A small <<column>> of liquid leaves the capillary and gives a droplet which freely flies in the air. Depending on the spout sizes (30 to 100 μM), volumes from 25 up to 500 μl_are produced allowing drops having diameters from 35 to 100 μm to be obtained.

Among all these techniques, it seems that only three may be contemplated for producing localized deposits of the sol-gel type on glass slides i.e. <<spray coating>> using a mask, drop deposit by <<Microdrop>> and <<contact printing>> however, these techniques can have drawbacks, notably when they are used for depositing solutions of the sol-gel type.

Also, international application WO 2008/040769 published on Apr. 10^{th} 2008 proposes a method for forming a film of polymer(s) notably selected from polyvinyl alcohol, poly(hydroxy)styrene, polyimide, polyethylene oxide and polyvinylcarbazole, on predetermined sites of a substrate. The proposed method consists in generating hydrophilic areas and hydrophobic areas on the substrate by using a photo-sensitive resin and photolithography. The polymer(s) is (are) then locally deposited on the hydrophilic areas by using techniques of localized and selective microdeposition of the piezo-electric actuator type, <<pin and ring>>, ink jet printing, Archimedean screw and micropipeting.

This method is applied to solutions of not very viscous and therefore strongly diluted polymer(s), which makes the drying step for removing the solvent necessary and mandatory.

For solutions of the sol type, the deposits obtained on a glass slide by using vaporization or manual spraying are not homogeneous and are reproducible with difficulty. This technique is therefore not suitable for a method involving solutions such as solutions capable of producing deposits of the sol-gel type.

During deposition tests with sol microdrops and notably by using the method of international application WO 2008/040769, the capillary became blocked. Different cleaning tests were attempted in order to unblock the capillary in vain and show the difficulty of working with this deposition technique for synthesizing a sol-gel. In order to get rid of the blocking problem in the capillary, a modification of the synthesis of the sol-gel may be contemplated, for example by dilution, but with the risk of modifying the morphology of the sol-gel, and therefore the performances of the obtained layer. Further, this technique does not give the possibility of obtaining very diverse deposit shapes which essentially appear as drops or circular pads, and the control of the thickness is not easy since it is obtained by accumulation of drops.

Finally, the contact printing technique imposes working on PDMS/sol-gel interactions. At each developed synthesis, a study of the feasibility and adhesion between the PDMS and the sol-gel has to be conducted. Further, when a PDMS stamp comes into contact with the surface of the substrate, it rapidly releases a few molecules having hydrophobicity. This contamination changes the wettability of a hydrophilic surface into a hydrophobic surface, which may generate modifications in the behavior of the sol-gel at the surface of the substrate.

There therefore exists a real need for a method which is easy to apply and reproducible in order to deposit in a pre-determined, localized and defined way, a material of the sol-gel type.

### DISCUSSION OF THE INVENTION

The present invention gives the possibility of solving the drawbacks of the methods from the state of the art. Indeed, the present invention proposes a method allowing the localized deposition of a material of the sol-gel type. Remarkably, the method of the present invention not only applies to any type of material of the sol-gel type but also to other materials, notably to any polymeric material. It should be emphasized that the method according to the invention is not affected by the nature and notably by the viscosity of the solution containing said material, or from which said material may be obtained.

The method according to the present invention allows a localized and well-defined deposition of the material. It also has good reproducibility.

Further, such performances do not require many costly steps since the method according to the invention is inexpensive and may be applied in series. For example, it may be used for producing chips made on a 200/300 mm wafer.

More particularly, the present invention proposes a method for producing a localized deposit with a defined shape of a material on the surface of the substrate, comprising the steps:
delimiting by photolithography on the surface of said substrate, at least one localized site with a defined shape, wettable with a solution containing said material or from which said material is obtained, the areas delimiting said site being non-wettable with said solution;
depositing, on said site and said areas, said solution;

whereby said material is deposited at said site directly on the substrate,

said site and said areas being coplanar on the surface of the substrate.

By <<localized deposit with a defined shape of a material at the surface of a substrate>>, is meant within the scope of the present invention, a deposit of the material on one (or more) predefined site(s), chemically materialized on the surface of the substrate.

Indeed, the method according to the invention comprises a first step which allows preparation, on the surface of the substrate, of one (or more) wettable site(s) with the solution containing the material of interest or from which this material is obtained, delimited, for example surrounded, by areas which are non-wettable with this solution. This first step applied before deposition of the solution therefore allows control of the surface on which is carried out the deposition.

This first delimitation step successively applies a photosensitive resin, a photolithography and a non-wettable material with the solution containing the material of interest or from which this material is obtained, i.e. a material having a hydrophilic or hydrophobic nature distinct from that of said material.

The thereby obtained substrate may either be used immediately for depositing the solution thereon, or be stored from two hours to twelve months and notably from six hours to six months before deposition of the solution is achieved. This storage possibility without the properties of the prepared substrate being affected, is another advantage of the method according to the invention. This storage possibility allows the user to have a substrate ready to be handled, once the material to be deposited has been selected and thus allows anticipation of the needs of the user.

The present invention is based on a wise choice of a substrate comprising sites having wettability towards the solution containing the material of interest or from which this material is obtained, while the areas delimiting and notably surrounding the sites are non-wettable towards the solution. By such a choice, it is thereby possible to obtain a material exclusively present at wettable site(s) and this even by depositing the solution containing the material of interest or from which this material is obtained, on the totality of the surface of the substrate i.e. both on the site(s) and on the areas as defined earlier. The present invention uses the wettability properties of the optionally pre-treated substrate, in order to select the solution containing the material of interest or from which this material is obtained.

Wettability is defined by the contact angle (or connecting angle) which a drop of the solution forms with the substrate at the deposition site of this drop.

Thus, when it is specified that the deposition sites are wettable towards the solution, this generally means that a drop of this deposited solution will form relatively to the deposition site a contact angle generally having a value of less than 70° and notably less than 60° while, for the non-wettable areas surrounding said site, this means that the contact angle formed between a drop of the solution and these areas generally has a value of more than 90° and notably more than 95°. From a practical point of view, this means that the solution, when it is deposited on the whole of the surface of the substrate, is concentrated at the wettable sites with this solution and does not remain or only very little at the non-wettable areas.

From a chemical point of view, a liquid will wet a solid substrate if it has chemical affinity towards the latter. Thus, a hydrophobic solid substrate will be wettable towards hydrophobic liquids and vice versa.

By <<deposited at said site directly on the substrate>>, is meant within the scope of the present invention, that there exists a direct contact between the substrate and the solution containing said material or from which said material is obtained and, finally between the substrate and the material. Thus, no sub-layer exists, separating the substrate from the solution and finally separating the substrate from the material. By <<substrate>> should be meant the substrate at the moment of the application of the method herein, the latter having been capable, before this application, of undergoing pre-treatment. The absence of a sub-layer or intermediate layer gives the possibility of avoiding possible interactions between the latter and the deposited material.

The first delimitation step gives the possibility of obtaining chemical structuration of the surface of the substrate by only acting on wettability. Indeed, this step does not apply any physical structuration involving relief elements of the microstructure type notably as described in patent application US 2003/148401. In other words, the wettable sites and the non-wettable areas towards the solution containing the material of interest or from which this material is obtained, present on the surface of the substrate, are coplanar.

The substrate applied within the scope of the present invention is a substantially planar solid substrate. This may be any substrate with which this invention may be applied. As a substrate example, mention may be made of a biochip support or a microscope slide such as those conventionally used, in silicon, in glass, in metal, in polymer or in plastic. It may be of various sizes and shapes.

The substrate may, before applying the method according to the invention, have undergone a preparatory treatment so as to modify and/or improve the hydrophilic or hydrophobic properties of its surface. This treatment may consist in chemical modification of the surface of the support with treatments such as oxidizing treatments or by depositing a coating on the surface with the usual deposition techniques known to one skilled in the art. This coating may for example be silicon, glass, silicon dioxide or a (per)fluorinated polymer.

Alternatively, the substrate may, before applying the method according to the invention, have not undergone any preparatory treatment in order to modify and/or improve the hydrophilic or hydrophobic properties of its surface.

The material deposited on said substrate may be a hydrophilic or hydrophobic material. Indeed, the method according to the present invention may be used whether the material to be deposited is hydrophilic or hydrophobic.

The material deposited on said substrate is advantageously a porous material. The porosity of a material gives the possibility of establishing a nomenclature according to the size of the pores. Indeed, according to the rules established by the International Union of Pure & Applied Chemistry (IUPAC), it is possible to distinguish, according to the average pore diameter in a material, micropores, (smaller than 20 Å), mesopores (20-500 Å) and macropores (greater than 500 Å). The material applied within the scope of the present invention is more generally microporous. Alternatively, the material applied within the scope of the present invention is mesoporous or macroporous. Whether the material is macroporous, mesoporous or microporous, it has a specific surface area from 200 to 800 m²·g⁻¹, notably from 300 to 700 m²·g⁻¹ and, in particular from 400 to 600 m²·g⁻¹.

In a first embodiment, the material deposited on the substrate according to the method of the invention is a polymeric material and advantageously a porous polymeric material.

By <<polymeric material>> is meant, within the scope of the present invention, a soluble or insoluble, natural or synthetic (co)polymer and notably organic, said material being advantageously porous. The polymeric material used in the present invention may be a hydrogel.

Thus, the polymeric material which may be used within the scope of the present invention is selected from agarose; gelatin; cellulose; carboxymethylcellulose; an alginate; a polyolefin; a styrenic polymer such as an advantageously porous polystyrene resin; a halogenated hydrocarbon polymer like polytetrafluoroethylene or poly(chloro-trifluoroethylene); a vinyl polymer such as poly(vinyl decanoate) or polyvinyl alcohol; a (meth)acrylic polymer such as poly(n-butyl acetate) or poly(benzyl methacrylate); polyethylene glycol; poly(propylene fumarate); poly(ethylene fumarate); poly(alpha-hydroxyester); poly(orthoester); polyanhydride; poly(phosphazene); poly(ester amide); polylactic acid; polyglycolic acid; polycaprolactone (PCL); polydioxanone (PDO); polyurethane; a gel of cholesteryl anthraquinone-2-carboxylate and of polymethylsiloxane; a gel of 1,3:2,4-dibenzylidene-sorbitol and of octamethylcyclotetrasiloxane; a gel of aromatic diamide with a perfluorinated chain and of perfluoro-tributylamine notably described in the article of Loiseau et al., 2002, Tetrahedron, Vol. 58, pages 4049-4052.

In a second embodiment, the material deposited on the substrate according to the method of the invention is a material of the sol-gel type.

By <<material of the sol-gel type>> or <<sol-gel material” both expressions being equivalent and interchangeable, is meant a material obtained by a sol-gel method consisting of using as precursors, metal alkoxides, either identical or different, of formula M(OR)ₙ(R′)ₘ wherein M is a metal such as silicon, R and R′ represent an alkyl group and m and n are integers with m+n=4, 2≦n≦4 et 0≦m≦2. The sol-gel materials are generally prepared in a solvent, which is preferably miscible with water and evaporable under mild conditions, in which solvent the precursors are soluble. In the case of silicon alkoxides, mention may notably be made of alcohols, such as methanol, ethanol; of ethers, such as diethyl ether and tetrahydrofurane; of chlorinated solvents, such as chloroform, CH₂Cl₂, C₂H₅Cl₂ or other aprotic solvents like CH₃CN, acetone, methylethylketone; or dioxane or protic solvents such as acetic acid, formamide. In the presence of water, hydrolysis of the alkoxide groups (—OR) occurs and the latter are transformed into silanol (Si—OH) groups which condense by forming siloxane groups (Si—O—Si). Small particles with a size generally less than 1 nanometer are then formed. They aggregate and form lacunal clusters suspended in the liquid; this is the sol. The polycondensation continuing over time, the viscosity of the sol increases until gelling: the sol becomes a gel. A solid sol-gel material is obtained by drying the gel. During this step, the residual and interstitial solvents escape from the formed polymeric lattice and evaporate which causes contraction of the material. A final material is obtained, the volume of which is reduced as compared to the volume occupied by the sol. The sol therefore corresponds to a solution from which the material of interest will be deposited, in this case the sol-gel material, is obtained.

The sol-gel material applied within the scope of the present invention is essentially prepared from 1 to 4 alkoxysilane precursors and essentially obtained from the hydrolysis of 1 to 4 alkoxysilane precursors. The sol-gel material applied within the scope of the present invention is therefore essentially made up of units stemming from the hydrolysis of a single alkoxysilane precursor or stemming from two, three or four different alkoxysilane precursors.

As an alkoxysilane precursor which may be used within the scope of the present invention, mention may be made of tetramethoxysilane (TMOS), tetraethoxysilane (TEOS), tetrapropoxysilane (TPOS), tetrabutoxysilane (TBOS), methyltrimethoxysilane (MTMOS), ethyl-trimethoxysilane (ETMOS), propyltrimethoxysilane (PTMOS), methyltriethoxysilane (MTEOS), ethyl-triethoxysilane (ETEOS), propyltriethoxysilane (PTEOS), 3-aminopropyltriethoxysilane (APTES), 3-aminopropyl-trimethoxysilane (APTMS), (3-(methylamino)propyl)-trimethoxysilane, 3-carboxypropyltriethoxysilane, 3-carboxypropyltrimethoxysilane, 1,2-bis(triethoxysilyl)-ethane, 1,2-bis(trimethoxysilyl)ethane, (3,3,3-tri-chloropropyl)triethoxysilane, 3,3,3-trifluoropropyl-trimethoxysilane and mixtures thereof.

Advantageously, the alkoxysilane precursor applied within the scope of the present invention is TMOS.

The sol-gel material may further contain structuring compounds such as organic polymers, like ionomers and notably fluorinated organic polymers derived from ethylene with an acid function, such as NAFION®, and also generally neutral surfactants.

The final sol-gel material generally contains at most 95% by mass of alkoxysilane derivatives, notably at most 85% by mass of alkoxysilane derivatives and in particular, from 60 to 80% by mass of alkoxysilane derivatives.

Finally, whether the material deposited on a substrate according to the method of the invention is a polymeric material or a material of the sol-gel type, it may further comprise at least one probe molecule. In other words, at least one probe molecule is incorporated to the material applied within the scope of the present invention.

By <<probe molecule>> is meant, within the scope of the present invention, a molecule specific to one (or more) analyte(s) for which the contacting with at least one of these analyte(s) causes at least one modification of the spectral properties of this probe molecule.

The probe molecule applied within the scope of the present invention is a molecule which has fluorogenic or chromogenic properties, i.e. it becomes fluorescent or is colored when it interacts with at least one specific analyte. Generally, the interaction of the probe molecule with at least one specific analyte produces a detectable optical signal. The interaction may consist in the creation of an irreversible and selective bond notably of the covalent bond type between the probe molecule and at least one specific analyte.

One skilled in the art is aware of different probe molecules which may be used within the scope of the present invention and are known for detecting specific analytes, either volatile or liquid such as an aldehyde, formaldehyde, acetaldehyde, naphthalene, a primary notably aromatic amine, indole, skatole, tryptophan, urobilinogen, pyrrole, benzene, toluene, xylene, styrene, naphthalene and volatile biomarkers such as 1-methyl naphthalene, p-methyl anisate, methyl nicotinate and o-phenyl anisole. Such probe molecules are notably selected from enaminomes and corresponding β-diketone/amine pairs, imines and hydrazines, 4-aminopent-3-en-2-one (Fluoral-P), croconic acid and probe molecules with an aldehyde function which are p-dimethylaminobenzaldehyde (DMABA or DAB), p-dimethylaminocinnamaldehyde (DMACA), p-methoxy-benzaldehyde (MOB) and 4-methoxy-1-naphthaldehyde (MON), mixtures and salts derived from these compounds. Additional information on the probe molecules which may be used, can be found in international application WO 2007/031657 published on Mar. 22^{nd} 2007.

When it comprises at least one probe molecule, the material applied within the scope of the present invention is preferably a porous material as defined earlier. Thus, the probe molecule(s) is(are) found at the surface of the pores of the polymeric material or of the material of the sol-gel type. The probe molecules may be adsorbed at the surface of the pores of this material and/or bound to this surface through non-covalent bonds (hydrogen bonds or ionic bonds) and/or through covalent bonds. Generally, the probe molecules are distributed in the whole of the volume of the material. The use of a porous material thereby allows control of the diffusion of notably gaseous analytes and promotion of their contacting with probe molecules.

Also, when the material applied within the scope of the present invention comprises at least one probe molecule, the substrate is advantageously transparent or translucent so as not to affect the detection of the signal emitted by the probe molecule in the presence of at least one specific analyte.

The weight percentage of probe molecules is advantageously from 0.01 to 30%, in particular, from 0.1 to 20% and, most particularly, from 1 to 10% based on the total weight of the porous polymeric or sol-gel type material.

The method according to the present invention more particularly comprises the steps:

a) depositing on the surface of said substrate a layer of photosensitive resin;

b) removing by photolithography, the resin layer in given areas, whereby the resin subsists on at least one localized site and with a defined shape, delimited and notably surrounded by said areas;

c) depositing, on said areas, a more hydrophobic or more hydrophilic compound than the surface of said substrate;

d) removing the subsisting photosensitive resin, whereby said localized site with a defined shape no longer has any resin at its surface;

e) depositing, on said site and said areas, a solution containing said material or from which said material is obtained, said site being wettable with said solution and said areas non-wettable with the latter.

The method according to the present invention has two alternatives based on the hydrophilic or hydrophobic nature of the surface of the substrate.

Indeed, in the case when the surface is hydrophilic, the solution containing the material to be deposited or from which this material is obtained, is also hydrophilic. This first alternative comprises the steps:

a₁) depositing on the hydrophilic surface of said substrate a layer of photosensitive resin;

b₁) removing by photolithography the resin layer in given areas, whereby the resin subsists on at least one localized site and with a defined shape, delimited and notably surrounded by said areas;

c₁) depositing, on said areas, a more hydrophobic compound than the surface of said substrate;

d₁) removing the subsisting photosensitive resin, whereby said localized site with a defined shape is more hydrophilic than the compound deposited in step (c₁);

e₁) depositing on said site and said areas, a hydrophilic solution containing said material or from which said material is obtained.

The second alternative relates to the case when the surface of the substrate and the solution containing the material to be deposited or from which this material is obtained, are hydrophobic. This second alternative comprises the steps:

a₂) depositing on the hydrophobic surface of said substrate, a layer of photosensitive resin;

b₂) removing by photolithography the layer of resin in given areas, whereby the resin subsists on at least one localized site and with a defined shape, delimited and notably surrounded by said areas;

c₂) depositing, on said areas, a more hydrophilic compound than the surface of said substrate;

d₂) removing the subsisting photosensitive resin, whereby said localized site with a defined shape is more hydrophobic than the compound deposited in step (c₂);

e₂) depositing, on said site and said areas, a hydrophobic solution containing said material or from which said material is obtained.

In the method according to the present invention and to its alternatives, the steps (a), (a₁) and (a₂) consist in depositing a thin layer of photosensitive resin on the surface of the substrate. It may be necessary, before this deposition step, to subject the surface of the substrate to an oxidizing treatment, or with an adhesion layer by means of an adhesion promoter such as HMDS (for hexamethyl-dimethylsiloxane). The goal of this preliminary step is to obtain better adhesion of the resin which will be applied subsequently. By <<thin layer>> is meant a layer having a substantially uniform thickness, comprised between 10 nm and 100 μm and notably between 50 nm and 20 μm. This deposition may be carried out with any technique with which a thin layer of resin may be obtained. Advantageously, said deposition is carried out by immersion (dip coating), by vaporization (spray coating) or by centrifugation (spin coating), the latter giving the possibility of spreading out a small amount of photosensitive resin on a substrate by means of centrifugal forces on a whirler.

The photosensitive resin applied within the scope of the present invention may be a so-called <<positive>> resin, i.e. a resin for which the insolated areas are removed by the chemical developer or a so-called <<negative>> resin i.e. a resin for which the non-insolated areas are removed by the chemical developer.

Any positive or negative photosensitive resin known to one skilled in the art may be used within the scope of the present invention. As non-limiting examples, mention may be made of the resin TELR-P0003PV (Tokyo Ohka Kogyo Co. Ltd) in propylene glycol monomethyl ether acetate, the resin SU-8 (Shell Chemical) based on an octofunctional epoxy with a triarylsulfonium salt as a photoinitiator or the resin of the Novolac type, based on phenolformaldehyde with diazonaphthoquinone (DNQ) as a photoinitiator.

Following the deposition of the photosensitive resin and before steps (b), (b₁) and (b₂), the resin may be heated to a temperature comprised between 80° C. and 125° C. and notably between 90° C. and 115° C. for a duration depending on the thickness of the layer and generally comprised between 1 and 30 mins. This curing step allows removal of the solvent.

Steps (b), (b₁) and (b₂) of the method according to the invention consist in irradiating the resin layer by means of UV radiation through a mask defining insolated areas and non-insolated areas and thereby the contour (or the limits) of the localized site(s) and with a defined shape and then in removing either the insolated areas or the non-insolated areas.

The localization, the size and the shape of the mask used define the localization, the size and the shape of the site(s) as defined earlier and therefore the localization, the size and the shape of the material deposit according to the method of the invention. By suitably selecting the mask used during steps (b), (b₁) and (b₂), the site(s) as defined earlier and therefore the material deposit(s) may appear as pads or spots having a diameter comprised between 1 μm and 5 cm or as strips, the length of which may attain up to 20 cm and the width of which is comprised between 1 μm and 2 cm. Any mask customarily used in photolithography may be used within the scope of the present invention. As non-limiting examples, such a mask may be made in quartz and/or in chromium.

Typically, the UV irradiation (or insolation) intensity is comprised between 100 and 1,500 mJ·cm² and notably between 200 and 1,000 mJ·cm². The UV irradiation may be performed for a duration comprised between 1 s and 2 mins and notably between 5 s and 1 min. If necessary, a curing step of the resin may be required for completing the photopolymerization induced by the UV irradiation. This curing step is advantageously performed between 80° C. and 110° C. and notably between 90° C. and 95° C. for 15 to 30 mins.

The insolated areas i.e. the photopolymerized areas or the non-insulating areas become insensitive to a large majority of solvents. On the other hand, the insolated areas for positive resins or the non-insolated for negative resins may subsequently be dissolved by a solvent, exposing the surface of the substrate at the areas as defined earlier. One skilled in the art depending on the photosensitive resin used is aware of the solvent, also called the developer, to be applied for removing certain areas of the resin after its UV irradiation. As non-limiting examples, mention may be made, as a solvent which may be used, of tetramethylammonium hydroxide (TMA 238), gamma butyro-lactone (GBL), propylene glycol methyl ethyl acetate (PGMEA), KOH or NaOH.

After the steps (b), (b₁) and (b₂) and prior to steps (c), (c₁) and (c₂), the resin may be subject to a post-curing step at a temperature comprised between 80° C. and 150° C. and notably between 90° C. and 130° C. and for a duration comprised between 30 s and 30 mins and notably between 1 and 10 mins.

Steps (c), (c₁) and (c₂) of the method according to the present invention consist in depositing on the surface of the substrate at the areas where the photosensitive resin has been removed during steps (b), (b₁) and (b₂), a more hydrophilic or more hydrophobic compound than the surface of the substrate.

This compound is advantageously selected from polytetrafluoroethylene like TEFLON®; silicon oxycarbide; a silane with a hydrophobic chain such as trichloromethylsilane (TCMS), trichloroethylsilane, trichloro(n-propyl)silane, trimethoxymethylsilane, triethoxymethylsilane, (3-phenylpropyl)-methyl-dichlorosilane (PMDS), benzyltrichlorosilane, methylbenzyltrichlorosilane, trifluoromethylbenzyl-trichlorosilane, methyltriethoxysilane, (3-phenyl-propyl)-methyldimethoxysilane, (3-phenylpropyl)-methyldiethoxysilane or (1H,1H,2H,2H)-perfluorodecyl-trichlorosilane (FDTS); or a silane with a hydrophilic chain such as 3-aminopropyltriethoxysilane, 3-aminopropylmethyldiethoxysilane, aminoethylamino-propylmethyldimethoxysilane, (N-phenylamino)methyl-trimethoxysilane, morpholinylpropyltrimethoxysilane, methyldimethoxysilane, dimethylethoxysilane, propyl-trimethoxysilane, butyltrimethoxysilane or dodecyl-trimethoxysilane.

The compound is deposited on areas as defined by any deposition technique known to one skilled in the art. However, in order to guarantee that this compound is maintained at these areas, the steps (c), (c₁) and (c₂) of the method according to the present invention consist in grafting this compound at the areas as defined earlier. By <<grafting>> is meant generating a covalent bond between the compound and the surface of the substrate, said bond involving an atom of the compound and an atom of the surface of the substrate. When the compound is a silane, this grafting step may consist in a silanization step.

It may be necessary, before this grafting step, to subject the surface of the substrate at the areas to an oxidizing treatment. By <<oxidizing treatment>>, is meant, within the scope of the present invention, a treatment (or pre-treatment) aiming at oxidizing the surface of the substrate applied and/or preparing the surface for future oxidation by forming radicals. An oxidation modifies the surface of the substrate, notably by attaching thereon and/or introducing thereon oxygen-rich groups such as groups of the carboxylic (—COOH), hydroxyl (—OH), alkoxyl (—OX with X representing an alkyl group, an acyl group or an aroyl group), carbonyl (—C═O), percarbonic (—C—O—OH), silanol (—SiOH) type and sometimes amide (—CONH) type.

Such an oxidation treatment is based on two main types of surface modifications based on:
physical treatments such as a plasma treatment notably with oxygen, a UV treatment, an X or gamma ray treatment, a treatment by irradiation with electrons and with heavy ions or
chemical treatments such as treatment with alcoholic potash, treatment with a strong acid (HCl, H₂SO₄, HNO₃, HClO₄), a treatment with sodium hydroxide, a treatment with a strong oxidizer (KMnO₄, K₂Cr₂O₇, KClO₃ or CrO₃ in hydrochloric acid, sulfuric acid or in nitric acid) and an ozone treatment.

Advantageously, the deposited compound during steps (c), (c₁) and (c₂) has a thickness comprised between 1 and 100 nm and notably between 2 and 50 nm.

The steps (d), (d₁) and (d₂) of the method according to the present invention consist in removing the photosensitive resin remaining in the site(s) as defined earlier. These steps require the use of a treatment and of one (or more) solution(s) or solvent(s) capable of removing the resin and therefore exposing the surface of the substrate at the sites, without removing the deposited compound and advantageously grafted at the areas of the substrate during steps (c), (c₁) and (c₂). Consequently, when the substrate is hydrophilic, the steps (d), (d₁) and (d₂) allow delimitation of one (or more) hydrophilic site(s) and vice versa when the substrate is hydrophobic.

One skilled in the art is aware of the treatments and solutions to be used depending on the resin to be removed. As examples, such a treatment may be accomplished with ultrasonic waves and by using one (or more) bath(s) in a solvent or in several either identical or different solvents such as acetone, methanol or ethanol.

Following the treatment of steps (d), (d₁) and (d₂), the substrate and more particularly the site(s) of the substrate at which the resin was removed during steps (c), (c₁) and (c₂) may be dried.

In fact, it is obvious that the sites and the substrate are in a same material, they are of the same chemical nature and therefore have the same chemical composition. During the application of the method, the surface of the substrate is not functionalized at the sites, unlike the surface at the areas. This lack of functionalization gives the possibility of avoiding chemical interactions with the solution containing the material or from which the material is obtained, and consequently avoiding possible chemical denaturation of the latter or of the obtained material.

Steps (e), (e₁) and (e₂) consist in depositing the material of interest at the site(s) as defined earlier. However, these steps are characterized by the fact that the solution containing this material or from which this material is obtained, is deposited not only at this(or these) site(s) but also at the areas delimiting it (or them) and notably surrounding it (or them). Thus, the applied deposition method during steps (e), (e₁) and (e₂) is advantageously selected from the group consisting of immersion deposition such as dip coating, vaporization deposition (spray coating), centrifugation deposition (spin coating) and deposition by coating. Generally, these are techniques for depositing a thin film, as opposed to techniques for deposition of microdrops. The deposition method during steps (e), (e₁) and (e₂) is more particularly a deposition by centrifugation (spin coating).

One skilled in the art depending on the nature of the polymer to be deposited is aware of the solution containing it or from which this material is obtained, to be used. This solution is defined as a liquid phase containing the polymer, the sol-gel or their precursors. Thus, the solution applied during steps (d), (d₁) and (d₂) may be a sol, a sol-gel being formed, a sol-gel, a solution in which the material is dissolved, a solution in which the material is suspended, an emulsion containing the material, a dispersion containing the material or a solution comprising the precursors of this material such as either identical or different monomers.

When the material of interest further comprises one (or more) probe molecule(s), it (they) may be incorporated to the material, after preparation of the latter. In this case, the incorporation may be accomplished by diffusion via a gas route by putting the probe molecule in gaseous form directly into contact with the material (with a partial vacuum or by circulation of the gas) or via a liquid route by putting the material directly into an (aqueous or solvent) solution containing the dissolved or diluted probe molecule. This incorporation may also be accomplished by functionalization or post-doping consisting in creating a covalent bond between the material and the probe molecule.

As a preferred alternative, the probe molecule(s) may be directly added into the solution containing the material or from which the solution is obtained, which causes direct encapsulation of the probe molecule in the material thereby allowing a better distribution of the probe molecules in the material.

The material deposit following steps (e), (e₁) and (e₂) may have a large volume, typically comprised between 50 μm³ and 200 mm³ and, in particular, between 100 μm³ and 5 mm³, with a thickness comprised between 30 nm and 100 μm and, in particular, between 100 nm and 5 μm.

Indeed, the method according to the present invention allows control of the thickness of the deposited material, the latter mainly depending on the viscosity of the solution containing the material or from which it is obtained. By controlling these parameters which are the viscosity of the solution and the parameters during the deposition of steps (e), (e₁) and (e₂), the method gives the possibility of depositing a reproducible material amount. The deposition of a reproducible material amount is particularly verified, during the use of a solution of the sol type since the method for preparing a solution of the sol type allows control of the viscosity thereof. Advantageously, the solutions used within the scope of the present invention are notably solutions of the sol type which have a viscosity comprised between 10⁻³ and 1 Pa·s (between 1 and 1,000 cp) and, in particular, between 2.10⁻³ and 0.1 Pa·s (between 2 and 100 cp), for shearing at 100 rpm and at room temperature (i.e. 21° C.±2° C.).

Finally, once the deposition of the solution has been carried out, the obtained material may be subject to post-treatment steps such as drying or notably thermal hardening of the curing type or under irradiation. However, unlike the method described in international application WO 2008/040769 published on Apr. 10^{th} 2008, the drying step is not mandatory. When it is applied and once the complete drying has been obtained, the materials of the sol-gel type deposited at the surface of the substrate may also be called <<monoliths>> or <<xerogels>>.

The present invention also relates to a substrate on the surface of which a material has been deposited in at least one localized site with a defined shape according to the method of a present invention, said material being a material of the sol-gel type as defined earlier. The material of the sol-gel type may further comprise a probe molecule as defined earlier. A particular example of such a substrate is a substrate having on its surface at least one deposit of a sol-gel material obtained from tetramethoxysilane (TMOS or tetramethylorthosilicate) and comprising Fluoral-P as a probe molecule. Such a material is described in international application WO 2007/031657 published on Mar. 22^{nd} 2007.

The present invention further relates on a substrate at the surface of which a material has been deposited in at least one localized site and with a defined shape according to the method of the present invention, said material being a polymeric material as defined earlier comprising at least one probe molecule as defined earlier.

Finally, the present invention relates to the use of such a substrate for trapping and/or detecting and optionally quantifying at least one chemical compound. In this case, the chemical compound is an analyte of the probe molecule incorporated into the material deposited on the substrate.

The present invention proposes a method for protecting a substrate consisting in forming on the surface of the substrate a deposit of a material, notably a polymeric material or of the sol-gel type according to the method of the invention. In this particular use of the method according to the invention, the sought protection may be protection against corrosion or against wear. Depending on the sought protection, one skilled in the art will be able to select the most suitable material for achieving this goal. As non-limiting examples of material which may be used, mention may for example be made of coatings based on SiO₂—ZrO₂, resistant to abrasion and which may be deposited on metals for protecting the latter against corrosion.

### SHORT DESCRIPTION OF THE DRAWINGS

FIG. 1 shows a schematization of the steps (a) and (b) of an alternative of a method according to the present invention applying a glass substrate, a hydrophilic material to be deposited of the sol-gel type containing a probe molecule and a positive photosensitive resin.

FIG. 2 shows a schematization of the steps (c) and (d) of the alternative of the method according to the present invention of FIG. 1.

FIG. 3 shows a schematization of the step (e) of the alternative of the method according to the present invention of FIG. 1, this step consisting in a deposition of the sol-gel material.

FIG. 4 shows photographs of two sol-gel deposits obtained according to the method of the present invention (FIGS. 4A and 4B).

### DETAILED DISCUSSION OF PARTICULAR EMBODIMENTS

### I. The Method According to the Present Invention

I.1. Preparation of localized photosensitive resin deposits.

The substrate used is a glass slide.

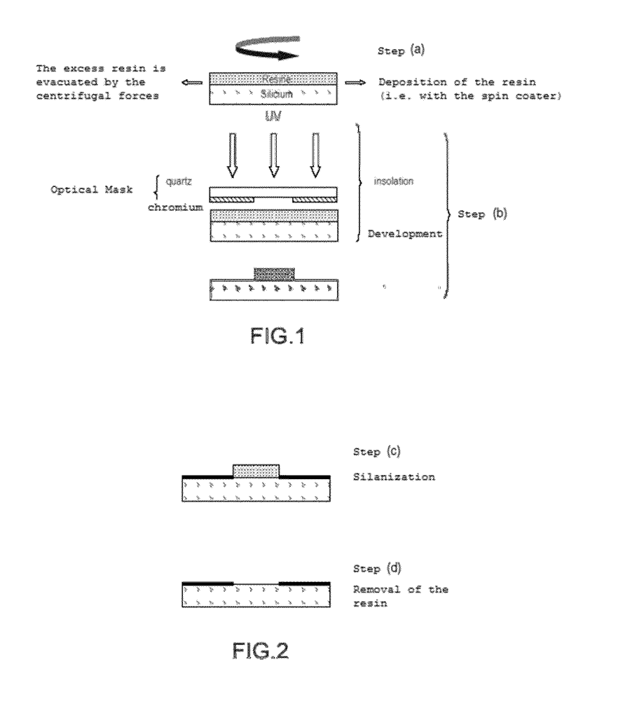

In this example, the resin used is TELR-P0003PV (propylene glycol monomethyl ether acetate, Tokyo Ohka Kogyo Co. Ltd) the viscosity of which is equal to 5 mPa·s (FIG. 1, step(a)). After depositing the resin by means of a spin coater, a curing step is required in order to rapidly remove a portion of the solvents and ensure polymerization of the matrix. This curing is carried out for 1 min at a temperature of 110° C.

The insolation consists in exposing certain areas of the resin via a masking system to ultraviolet radiation (FIG. 1, step (b)). The mask applied is a photolithographic mask made in quartz and in chromium. The insolation is carried out with a UV lamp of the MA8 type, the insolation time is 20 s at a power of 390 W.

The resin is then developed by means of a developer TMA 238 provided by JSR. This developer is a basic aqueous solution (normality 0.28 N) conventionally containing tetramethylammonium hydroxide, KOH and NaOH. It allows the removal of the insolated resin (FIG. 1, step (b)). Then a curing step is required in order to remove the residual solvents and cross-link the resin. This curing is carried out for 2 mins at 130° C.

I.2. Preparation of the Hydrophobic Pads.

After curing, the substrate i.e. the glass slide, still has hydrophilic properties (contact angle (water)=35-40°. Thus, a silanization step (FIG. 2, steps (c)) is considered in order to make the substrate hydrophobic. Before this silanization, the substrate is treated with oxygen plasma (1 min, power of 600 W, with the Plassys equipment) in order to create at its surface, silanol groups.

Silanization is achieved with MVD (Molecular Vapor Deposition) with, as a silane, (1H,1H,2H,2H)-perfluorodecyl-trichlorosilane (FDTS) of formula:

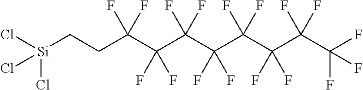 [SMILES: FC(F)(F)C(F)(F)C(F)(F)C(F)(F)C(F)(F)C(F)(F)C(F)(F)C(F)(F)CC[Si](Cl)(Cl)Cl]

In this step, a deposit of a few nm of silane is achieved on the substrate. The drop angle is thus of the order of 108-110°.

The resin is then removed (FIG. 2 step (d)). To do this, the substrate is rinsed for 10 mins with acetone with ultrasonic waves, 10 mins with ethanol with ultrasonic waves and 10 mins with water with ultrasonic waves and then dried with the centrifuge for 10 mins at 1,000 rpm. Following the rinsing operations, the deposited silane is not removed. The resin is thus removed and, at the location where the resin was localized, the substrate is hydrophilic.

I.3. Localized Deposits of Sol-Gel Material.

A sol-gel material comprising 4-amino-3-penten-2-one (Fluoral-P) is then deposited on the hydrophilic areas of the substrate.

This material doped with Fluoral-P is obtained from a siliceous precursor, tetramethoxysilane (TMOS). Initially, 100 mg of Fluoral-P are dissolved with sonication in 1,030 μL of ethanol of spectroscopic quality. 651 μL of TMOS and 318 μL of Millipore water (R=18 MΩ) are then added.

The thereby obtained 2 mL of sol are kept in a hermetically sealed (plug+parafilm) pillbox. The sol is then sonicated again for 15 mins in order to avoid aggregation of the Fluoral-P molecules, and mechanically stirred until deposition.

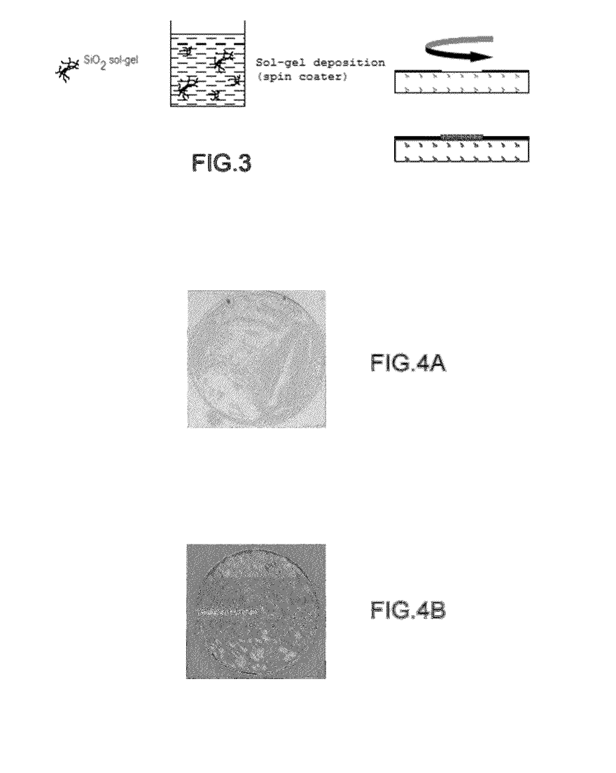

Deposition of the sol is carried out by spin coating: deposition time 1 min, speed of 2,000 revolutions per minute. During the deposition of the sol over the whole surface of the substrate, i.e. on the hydrophilic pads and hydrophobic pads encircling these hydrophilic areas (FIG. 3), the formed sol-gel is concentrated on the hydrophilic portions of the substrate, while occupying all the possible space. Thus, on the hydrophobic portions, no trace amount of sol-gel is present.

In this way, deposits of sol-gel are obtained, the shape of which is well defined and the thickness is well controlled by the conventional deposition method with the spin coater (FIG. 4).

### II. SOL-GEL PADS OBTAINED BY THE METHOD ACCORDING TO I

FIG. 4A is a photograph of a sol-gel pad obtained according to the method described in point I, with a substantially round shape, having a diameter of 2 mm and a thickness comprised between 160 and 180 nm.

FIG. 4B is a photograph of another sol-gel pad obtained according to the method described in point I, with a substantially round shape, having a diameter of 1 mm and a thickness comprised between 560 and 580 nm.

The sol-gel material obtained by applying the method described in point 1.3 has a specific surface area of 519±50 m²·g⁻¹ and a micropore surface of 85.9±5%.

1-12. (canceled)

13. A method for producing a localized deposit of a material which is localized and has a defined shape on the surface of a substrate, comprising the steps:
delimiting by photolithography on the surface of said substrate, at least one localized site and with a defined shape, wettable with a solution containing said material or from which said material is obtained, the areas delimiting said site being non-wettable with said solution;
depositing, on said site and said areas, said solution;
whereby said material is deposited at said site directly on said substrate,
said site and said areas being coplanar on the surface of the substrate.

14. The method according to claim 13, wherein said material is porous.

15. The method according to claim 13, wherein said material is a polymeric material.

16. The method according to claim 13, wherein said material is a material of the sol-gel type.

17. The method according to claim 13, wherein said material comprises at least one probe molecule.

18. The method according to claim 15, wherein said material comprises at least one probe molecule.

19. The method according to claim 16, wherein said material comprises at least one probe molecule.

20. The method according to claim 13, said method comprising the steps:
a) depositing on the surface of said substrate a photosensitive resin layer;
b) removing by photolithography the resin layer in given areas whereby the resin subsists on at least one localized site and with a defined shape delimited by said areas;
c) depositing on said areas, a more hydrophobic or more hydrophilic compound than the surface of said substrate;
d) removing the subsisting photosensitive resin whereby said localized site and with a defined shape no longer has any resin at its surface;
e) depositing, on said site and said areas, a solution containing said material or from which said material is obtained, said site being wettable with said solution and said areas non-wettable with the latter.

21. The method according to claim 20, wherein said step (c) consists in grafting said compound at said areas.

22. The method according to claim 13, wherein said deposition, on said site and said areas, of said solution is selected from the group consisting of deposition by immersion such as dip coating, deposition by vaporization (spray coating) deposition by centrifugation (spin coating) or deposition by coating.

23. A substrate on the surface of which a material was deposited in at least one localized site and with a defined shape according to a method as defined in claim 13,
said material being a material of the sol-gel type.

24. A substrate on the surface of which a material was deposited in at least one localized site and with a defined shape according to a method as defined in claim 13,
said material being a polymeric material comprising at least one probe molecule.

25. Method for trapping and/or detecting and optionally quantifying at least one chemical compound consisting in putting into contact said chemical compound into contact with a substrate according to claim 23.

26. Method for trapping and/or detecting and optionally quantifying at least one chemical compound consisting in putting into contact said chemical compound into contact with a substrate according to claim 24.

27. A method for protecting a substrate consisting in forming on the surface of the substrate a deposit of a material according to a method as defined in claim 13.
